(19) United States
(12) Patent Application Publication
(10) Pub. No.: US 2025/0148815 A1
(43) Pub. Date: May 8, 2025

(54) GRADIENT-TO-PARAMETER RATIO GUIDED FEATURE ALIGNMENT FOR MODEL ADAPTATION

(71) Applicant: NEC Laboratories America, Inc., Princeton, NJ (US)

(72) Inventors: Eric Cosatto, Red Bank, NJ (US); Evgenia Tatiani Chroni, Highland Park, NJ (US)

(21) Appl. No.: 18/938,766

(22) Filed: Nov. 6, 2024

Related U.S. Application Data
(60) Provisional application No. 63/596,999, filed on Nov. 8, 2023.

(51) Int. Cl. G06V 20/69; G06T 7/00; G16H 30/40; G16H 50/20

(52) U.S. Cl. CPC G06V 20/698; G06T 7/0012; G16H 30/40; G16H 50/20; G06T 2207/30024

(57) ABSTRACT

Systems and methods for gradient-to-parameter ratio guided feature alignment for model adaptation. To adapt an artificial intelligence (AI) model to different domains, activation statistics for the AI model can be computed from collected domain data. Weights of the AI model can be adjusted based on the activation statistics of the training gradients. The AI model can be fine-tuned by focusing adaptation intensity to layers with attention mechanism by using a ratio of gradient norm over parameter norm to obtain a fine-tuned AI model. The fine-tuned AI model can be employed to perform downstream tasks such as cell segmentation from medical images.

### RELATED APPLICATION INFORMATION

This application claims priority to U.S. Provisional App. No. 63/596,999, filed on Nov. 8, 2023, incorporated herein by reference in its entirety.

### BACKGROUND

### Technical Field

The present invention relates to adapting artificial intelligence models to different domains and more particularly to gradient-to-parameter ratio guided feature alignment for model adaptation.

### Description of the Related Art

Artificial intelligence (AI) models have improved dramatically over the years especially in entity detection, scene reconstruction, trajectory generation, and scene understanding. However, the accuracy of the AI models are directly proportional to the quality of data that they are trained with. A minor difference in obtaining the training dataset can have a major impact on the quality of training as AI models would treat such training dataset as a different domain. Training an AI model to understand such differences can lead to tedious data gathering as different domain require different training datasets.

### SUMMARY

According to an aspect of the present invention, a computer-implemented method is provided for adapting artificial intelligence (AI) models to different domains, including, computing activation statistics for an AI model from collected domain data, adjusting weights of layers of the AI model based on the activation statistics by employing metrics of training gradients, fine-tuning the AI model by focusing adaptation intensity to layers with attention mechanism by using a ratio of gradient norm over parameter norm to obtain a fine-tuned AI model, and performing downstream tasks with healthcare images using the fine-tuned AI model.

According to another aspect of the present invention, a system is provided for adapting artificial intelligence (AI) models to different domains, including a memory device, one or more processor devices operatively coupled with the memory device to compute activation statistics for an AI model from collected domain data, adjust weights of layers of the AI model based on the activation statistics by employing metrics of training gradients, fine-tune the AI model by focusing adaptation intensity to layers with attention mechanism by using a ratio of gradient norm over parameter norm to obtain a fine-tuned AI model, and perform downstream tasks with healthcare images using the fine-tuned AI model.

According to yet another aspect of the present invention, a non-transitory computer program product is provided including a computer-readable storage medium having program code for adapting artificial intelligence (AI) models to different domains, wherein the program code when executed on a computer causes the computer to compute activation statistics for an AI model from collected domain data, adjust weights of layers of the AI model based on the activation statistics by employing metrics of training gradients, fine-tune the AI model by focusing adaptation intensity to layers with attention mechanism by using a ratio of gradient norm over parameter norm to obtain a fine-tuned AI model, and perform downstream tasks with healthcare images using the fine-tuned AI model.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a flow diagram illustrating a high-level overview of a method for adapting an AI model to different domains with gradient-to-parameter ratio guided feature alignment, in accordance with an embodiment of the present invention;

FIG. 2 is a block diagram illustrating a system implementing a practical application of gradient-to-parameter ratio guided feature alignment for model adaptation, in accordance with an embodiment of the present invention;

FIG. 3 is a flow diagram illustrating a computing system device adapting an AI model to different domains with gradient-to-parameter ratio guided feature alignment, in accordance with an embodiment of the present invention;

FIG. 4 is a block diagram showing a software implementation with hardware components of adapting an AI model to different domains with gradient-to-parameter ratio guided feature alignment, in accordance with an embodiment of the present invention; and

FIG. 5 is a block diagram illustrating adapting computation layers of a deep neural network to different domains, in accordance with an embodiment of the present invention.

### DETAILED DESCRIPTION OF PREFERRED EMBODIMENTS

In accordance with embodiments of the present invention, systems and methods are provided for gradient-to-parameter ratio guided feature alignment for model adaptation.

In an embodiment, an artificial intelligence (AI) model can be adapted to different domains. To adapt the AI model to different domains, activation statistics can be calculated for the AI model from collected domain data. Weights of the AI model can be adjusted based on the gradients by employing metrics of the training gradients. The AI model can be fine-tuned by focusing adaptation intensity to layers with attention mechanism by using a ratio of gradient norm over parameter norm to obtain a fine-tuned AI model. The fine-tuned AI model can be employed to perform downstream tasks such as cell segmentation from medical images.

The present invention can adapt a trained neural network model to a new data domain without access to the training data. In many applications, such as medical applications, it is desirable to be able to adjust a trained model to a new data domain that is shifted from the one used in training the model. Typically, if no adaptation is done to the model, one can observe a clear degradation of performance. Such domain shift may exist in many different areas. For example, in medical histology images, different medical institutions generate pathology slides using specific protocols and reagent, resulting in subtle but distinct differences in colors and image quality. A model trained on data from one institution would typically perform poorly on the data from a different institution.

Obtaining a corpus of data to train such models requires significant logistical efforts as well as patient consent and generally carries privacy-preserving clauses that may preclude further use of the data down the line.

It is therefore desirable to develop a method by which the trained model can still be adapted at a later stage, should its application be needed in a new environment where a domain shift is present. In this invention, the present embodiments only require that the trained model is accompanied by statistics from the training of each of its layer. Those statistics are first order statistics of the training gradients (mean and variance) at each layer of the model, and therefore do not affect the privacy-preserving clauses of the layers.

Thus, the present embodiments can adapt a pre-trained neural network to different domains while preserving privacy clauses of the layers even without access to training data. By doing so, the present embodiments improve the accuracy of pre-trained neural networks, and in turn, the accuracy of the downstream tasks performed using such pre-trained neural networks.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

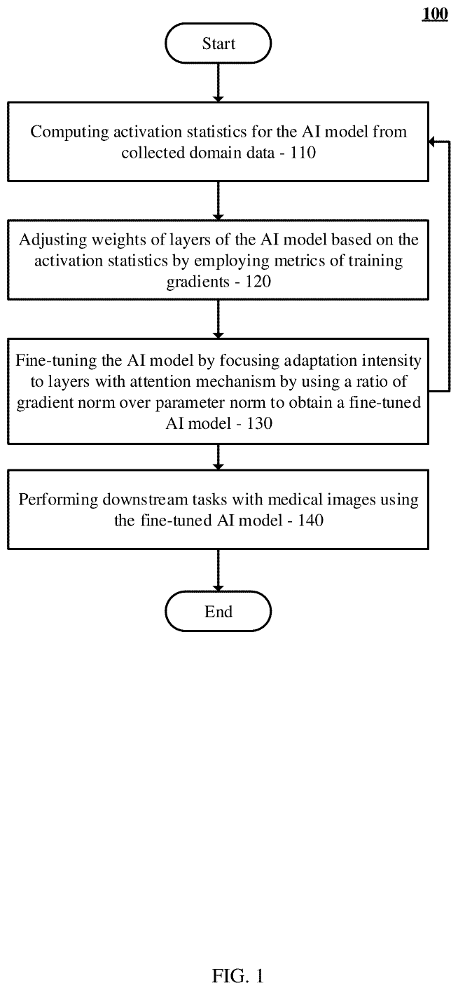

Referring now in detail to the figures in which like numerals represent the same or similar elements and initially to FIG. 1, a high-level overview of a computer-implemented method for adapting an AI model to different domains with gradient-to-parameter ratio guided feature alignment is illustratively depicted in accordance with one embodiment of the present invention.

In an embodiment, an artificial intelligence (AI) model can be adapted to different domains. To adapt the AI model to different domains, activation statistics for the AI model can be computed from collected domain data. Weights of the AI model can be adjusted based on the activation statistics of the training gradients. The AI model can be fine-tuned by focusing adaptation intensity to layers with attention mechanism by using a ratio of gradient norm over parameter norm to obtain a fine-tuned AI model. The fine-tuned AI model can be employed to perform downstream tasks such as cell segmentation from medical images.

Referring now to block 110 of FIG. 1 showing an embodiment of a method of computing activation statistics for the AI model from collected domain data.

Initially a multi-layer neural network model is trained on a corpus of data S. During training, activation statistics (e.g., mean and variance) at each layer are recorded. The output of the layers can be used by conducting a forward pass through the AI model with frozen parameters θ*. To compute the activation statistics, the following equations can be used with the following equation:

$${\hat{μ}}_{k} = {μ}_{k} ( S ; θ \text{*)} = \frac{1}{{N}_{s}} {\sum }_{x ∈ S} {Z}_{k} ( x ; θ \text{*)} ,$$

$${{\hat{σ}}_{k}^{2} = {σ}_{k}^{2} ( S ; θ \text{*)} = \frac{1}{{N}_{s}} {\sum }_{x ∈ S} ( {Z}_{k} ( x ; θ * ) {μ}_{k} ( S ; θ \text{*)} )}^{2}$$

where Zₖ (x;θ *) is a feature map of the selected layers k of the, \hat{μ}ₖ is the mean statistic, and \hat{σ}ₖ² is the variance statistic, Ns is the number of samples.

Referring now to block 120 of FIG. 1 showing an embodiment of a method of adjusting weights of layers of the AI model based on the activation statistics by employing metrics of the training gradients.

The layers recorded with the activation statistics can have their weights adjusted based on the activation statistics by employing metrics of the training gradients. The metrics of the training gradients can include source data statistics (e.g., mean and variance), target data statistics (e.g., mean and variance). To adjust their weights, for each layer k, a layer-wise loss can be minimized. The layer-wise loss can be computed as the distance between the source data statistics, \hat{μ}ₖ and \hat{σ}ₖ², and the target data statistics μₖ and σₖ² each batch B from a target data with the equation:

$${L}_{k} ( B ; θ ) = \vert {μ}_{k} ( B ; θ ) - {\hat{μ}}_{k} \vert + \vert {σ}_{k}^{2} ( B ; θ ) - {\hat{σ}}_{k}^{2} \vert ,$$

where |.| is the L1-norm, θ is the parameter of layer k.

Additionally, to optimize the network parameters and adjust the weights of the layers of the AI model by minimizing the average of the layer-wise loss, the following equation can be employed:

$${Loss}_{FA} = \frac{1}{K} {\sum }_{k ∈ K} {L}_{k} ( B ; θ ) ,$$

where K is the total number of layers recorded, Lₖ (B; θ) is the layer-wise loss for layer k.

Referring now to block 130 of FIG. 1 showing an embodiment of a method of fine-tuning the AI model by focusing adaptation intensity to layers with attention mechanism by using a ratio of gradient norm over parameter norm to obtain a fine-tuned AI model.

To standardize automatically weighing the importance of each term of the layer-wise loss the gradient norm (gₖ) to parameter norm (θₖ) ratio can be employed:

$$G 2 {P}_{k} = \frac{\vert \vert {g}_{k} {\vert \vert}_{2}}{\vert \vert {θ}_{k} {\vert \vert}_{2}}$$

To fine-tune the AI model, an alignment loss can be minimized to adapt to the data distribution of the target domain that is weighted by the respective gradient to parameter norm ratio. To minimize the alignment loss, the following equation can be employed:

$${Loss}_{G 2 P - FA} = \frac{1}{K} {\sum }_{k ∈ K} G 2 {P}_{k} {L}_{k} ( B ; θ ) .$$

The adaptation process described herein can be iteratively done for each layer of the AI model as processed for each batch of the target dataset. This can be summarized by the following pseudocode:
for B in target data do
for k in layers do
compute layer-wise loss for layer k, for batch B
backpropagate the layer-wise loss
compute the gradient to parameter norm ratio
end for
compute the alignment loss for layer k, and batch B
backpropagate the alignment loss
optimize the parameters of the model
end for

Referring now to block 140 of FIG. 1 showing an embodiment of a method of performing downstream tasks from medical images using the fine-tuned AI model.

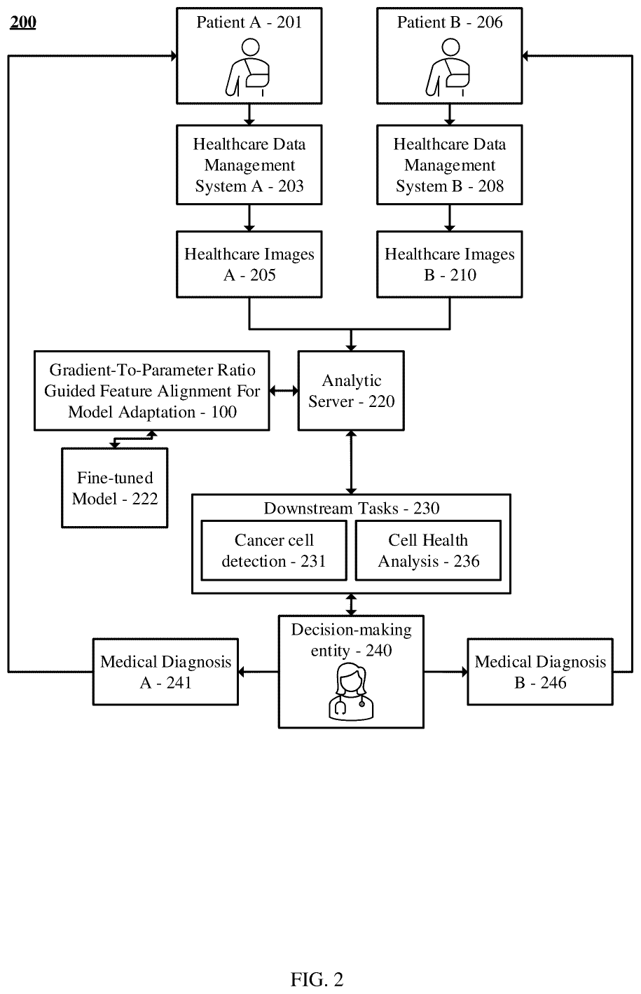

The downstream tasks that can be performed from medical images using the fined-tuned AI model can include cell/nuclei segmentation, cell detection, cell classification, cancer detection, etc. This is described in more detail in FIG. 2.

Referring now to FIG. 2 showing a block diagram of a system implementing a practical application of gradient-to-parameter ratio guided feature alignment, in accordance with an embodiment of the present invention.

System 200 shows a practical application in a healthcare setting. Different patient data can include different domains as the amount of data and type of data can differ between two patients and the methods and modalities used for their healthcare data.

Patient A 201 can provide healthcare data to a healthcare data management system (HDMA) A 203 which can generate healthcare images A 205. HDMA A 203 can employ image protocol A to obtain healthcare images A 205.

Patient B 206 can provide healthcare data to a HDMA B 208 which can generate healthcare images B 210. HDMA B 208 can employ image protocol B to obtain healthcare images B 210.

Because of the difference in image protocols, healthcare image B 210 and healthcare image A 205 are in different domains and an artificial intelligence (AI) model that would be trained to perform downstream tasks on each datasets would need different datasets. However, with the present embodiments, both domains can be adapted to an AI model with gradient-to-parameter ratio guided feature alignment 100 even without access to training data.

Healthcare images A 205 and healthcare images B 210 can be sent to an analytic server 220 through a network. The network can include a cloud computing environment, and other network implementations can be employed. The analytic server 220 can implement gradient-to-parameter ratio guided feature alignment 100 to obtain a fine-tuned model 222. The fine-tuned model 222 can be employed to perform downstream tasks 230 that includes cancer cell detection 231 and cell health analysis 236. The fine-tuned model 222 can be trained to determine relevant cells, a cancerous cell, whether a cell is benign or malignant for cancer cell detection 231. The fine-tuned model 222 can be trained determine relevant cells, whether a cell is healthy or unhealthy for cell health analysis 236.

Patient A 201 can request cancer cell detection 231 for his/her healthcare images A 205. The output of the cancer cell detection 231 can include proposed classifications of individual cells with annotations that can include descriptions of whether the cells are benign or malignant. This output can be sent to a decision-making entity 240 (e.g., healthcare provider) to update a medical diagnosis A 241 of Patient A 201.

Patient B 206 can request cell health analysis 236, such as sperm health analysis, for his healthcare images B 210. The output of cell health analysis 236 can include proposed classifications of individual cells with annotations that can include descriptions of whether the cells are healthy or unhealthy. This output can be sent to a decision-making entity 240 (e.g., healthcare provider) to update a medical diagnosis B 246 of Patient B 206.

Note that system 200 is not limited to the healthcare setting and can be implemented to other technical fields such as traffic scene understanding, reconstruction, trajectory generation, anomaly detection, etc.

Thus, the present embodiments can adapt a pre-trained neural network to different domains while preserving privacy clauses of the layers even without access to training data. By doing so, the present embodiments improve the accuracy of pre-trained neural networks, and in turn, the accuracy of the downstream tasks performed using such pre-trained neural networks.

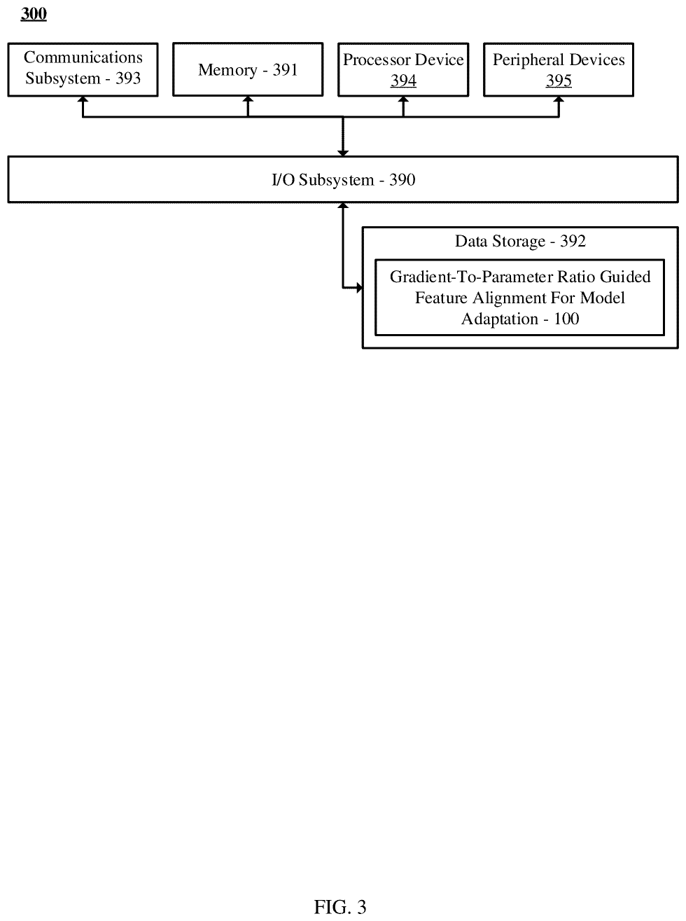

Referring now to FIG. 3, a computing system device for adapting an AI model to different domains with gradient-to-parameter ratio guided feature alignment is illustratively depicted in accordance with an embodiment of the present invention.

The computing device 300 illustratively includes the processor device 394, an input/output (I/O) subsystem 390, a memory 391, a data storage device 392, and a communication subsystem 393, and/or other components and devices commonly found in a server or similar computing device. The computing device 300 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 391, or portions thereof, may be incorporated in the processor device 394 in some embodiments.

The processor device 394 may be embodied as any type of processor capable of performing the functions described herein. The processor device 394 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 391 may be embodied as any type of volatile or non-volatile memory or data storage capable of performing the functions described herein. In operation, the memory 391 may store various data and software employed during operation of the computing device 300, such as operating systems, applications, programs, libraries, and drivers. The memory 391 is communicatively coupled to the processor device 394 via the I/O subsystem 390, which may be embodied as circuitry and/or components to facilitate input/output operations with the processor device 394, the memory 391, and other components of the computing device 300. For example, the I/O subsystem 390 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 390 may form a portion of a system-on-a-chip (SOC) and be incorporated, along with the processor device 394, the memory 391, and other components of the computing device 300, on a single integrated circuit chip.

The data storage device 392 may be embodied as any type of device or devices configured for short-term or long-term storage of data such as, for example, memory devices and circuits, memory cards, hard disk drives, solid state drives, or other data storage devices. The data storage device 392 can store program code for gradient-to-parameter ratio guided feature alignment for model adaptation 100. Any or all of these program code blocks may be included in a given computing system.

The communication subsystem 393 of the computing device 300 may be embodied as any network interface controller or other communication circuit, device, or collection thereof, capable of enabling communications between the computing device 300 and other remote devices over a network. The communication subsystem 393 may be configured to employ any one or more communication technology (e.g., wired or wireless communications) and associated protocols (e.g., Ethernet, InfiniBand®, Bluetooth®, Wi-Fi®, WiMAX, etc.) to affect such communication.

As shown, the computing device 300 may also include one or more peripheral devices 395. The peripheral devices 395 may include any number of additional input/output devices, interface devices, and/or other peripheral devices. For example, in some embodiments, the peripheral devices 395 may include a display, touch screen, graphics circuitry, keyboard, mouse, speaker system, microphone, network interface, and/or other input/output devices, interface devices, GPS, camera, and/or other peripheral devices.

Of course, the computing device 300 may also include other elements (not shown), as readily contemplated by one of skill in the art, as well as omit certain elements. For example, various other sensors, input devices, and/or output devices can be included in computing device 300, depending upon the particular implementation of the same, as readily understood by one of ordinary skill in the art. For example, various types of wireless and/or wired input and/or output devices can be employed. Moreover, additional processors, controllers, memories, and so forth, in various configurations can also be utilized. These and other variations of the computing system 300 are readily contemplated by one of ordinary skill in the art given the teachings of the present invention provided herein.

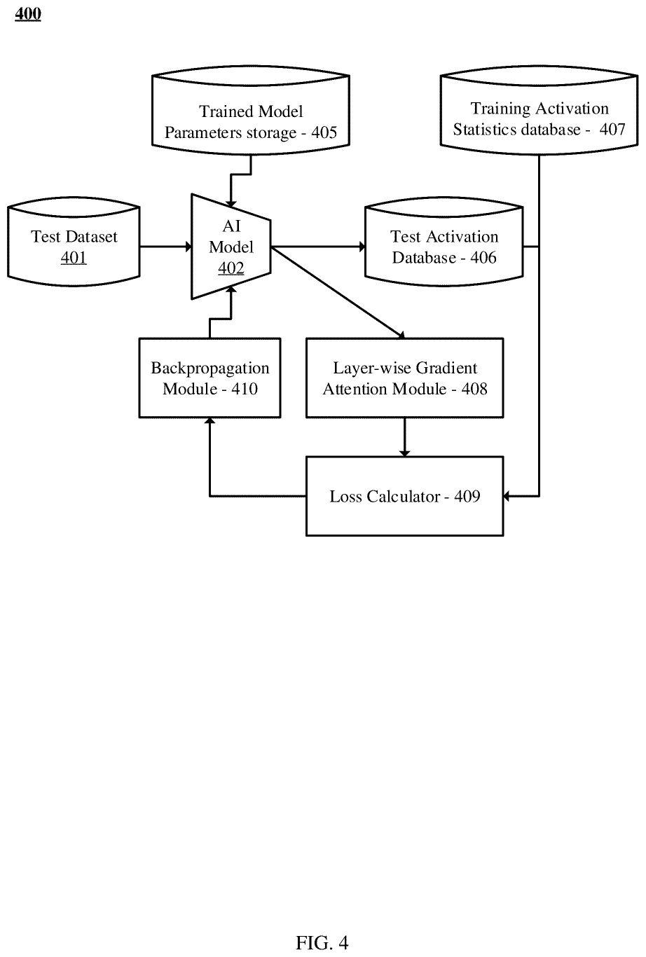

Referring now to FIG. 4, a block diagram showing a software implementation with hardware components of adapting an AI model to different domains with gradient-to-parameter ratio guided feature alignment, in accordance with an embodiment of the present invention.

System 400 can include hardware components AI model 402, Test activation database 406, activation statistics database 407, and trained model parameters storage 405.

To adapt the AI model 402 to different domains, AI model 402 can be initialized with trained model parameters from the trained model parameters storage 405. This adaptation phase can be run iteratively to adjust the AI model 402 to minimize the statistical differences between training activations stored in the training activation statistics database 407 and test activations stored in the test activation database 406. The test activations can be generated over a batch of test data from the test dataset 401.

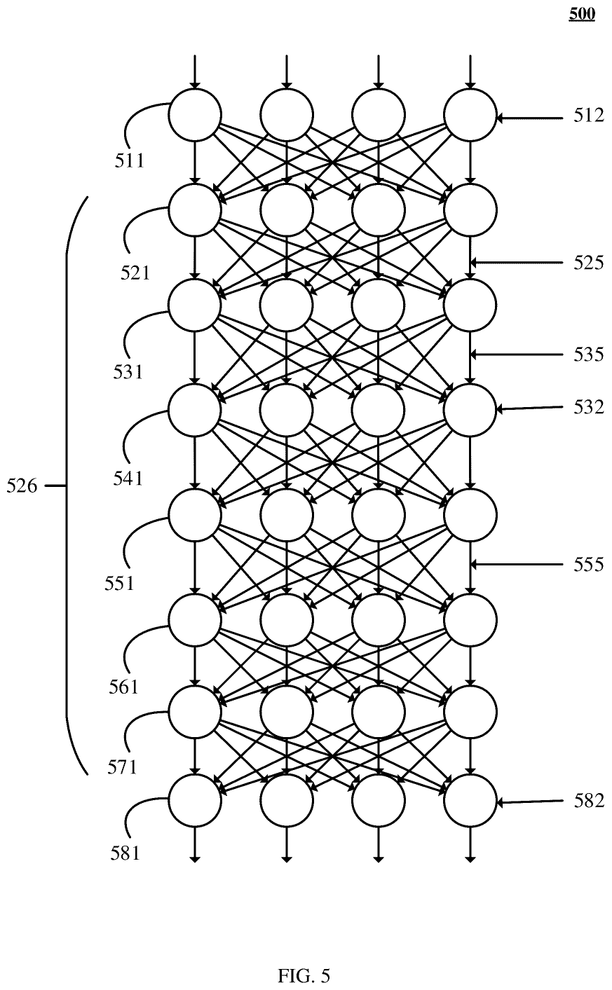

Both test and training activations can be obtained, weighted, and adjusted at each layer of the AI model 402 by utilizing the layer-wise gradient attention module 408 to obtain adjusted activations. This is shown in more detail in FIG. 5.

The weighing factor is based on the ratio of the gradient to the parameters obtained from the AI model 402. A loss function can then be calculated by the loss calculator 409 using the adjusted activations, the test activations and the training activations. The loss function would be used by the backpropagation module to backpropagate the loss function to fine-tune the AI model 402.

Referring now to FIG. 5, showing a block diagram of adapting computation layers of a deep neural network to different domains, in accordance with an embodiment of the present invention.

A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the inputted data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types and may include multiple distinct values. The network can have one input neurons for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

The deep neural network 500, such as a multilayer perceptron, can have an input layer 511 of source neurons 512, one or more computation layer(s) 526 having one or more computation neurons 532, and an output layer 581, where there is a single output neuron 582 for each possible category into which the input example could be classified. An input layer 511 can have a number of source neurons 512 equal to the number of data values 512 in the input data 511. The computation neurons 532 in the computation layer(s) 526 can also be referred to as hidden layers, because they are between the source neurons 512 and output neuron(s) 582 and are not directly observed. Each neuron 532, 542 in a computation layer generates a linear combination of weighted values from the values output from the neurons in a previous layer, and applies a non-linear activation function that is differentiable over the range of the linear combination. The weights applied to the value from each previous neuron can be denoted, for example, by w₁, w₂, . . . wₙ₋₁, wₙ. The output layer provides the overall response of the network to the inputted data. A deep neural network can be fully connected, where each neuron in a computational layer is connected to all other neurons in the previous layer, or may have other configurations of connections between layers. If links between neurons are missing, the network is referred to as partially connected.

Training a deep neural network can involve two phases, a forward phase where the weights of each neuron are fixed and the input propagates through the network, and a backwards phase where an error value is propagated backwards through the network and weight values are updated. The computation neurons 532 in the one or more computation (hidden) layer(s) 526 perform a nonlinear transformation on the input data 512 that generates a feature space. The classes or categories may be more easily separated in the feature space than in the original data space.

In an embodiment, computation layers 526 can include convolutional layers A 521, residual unit layer A 531, residual unit layer B 541, residual unit layer B 551, residual unit layer C 561, convolutional layer B 571. A layer-wise loss function A 525 can be generated from the gradients and parameters of convolutional layer A 521. A layer-wise loss function B 535 can be generated from the gradients and parameters of residual unit layer A 531. A layer-wise loss function C 555 can be generated from the gradients and parameters of residual unit layer B 551. The loss functions can then be used for the backpropagation step of deep neural network 500. To fine tune the deep neural network 500, an alignment loss that includes the layer-wise loss functions 525, 535, and 555 can be minimized.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for adapting artificial intelligence (AI) models to different domains, comprising:
computing activation statistics for an AI model from collected domain data;
adjusting weights of layers of the AI model based on the activation statistics by employing metrics of training gradients;
fine-tuning the AI model by focusing adaptation intensity to layers with attention mechanism by using a ratio of gradient norm over parameter norm to obtain a fine-tuned AI model; and
performing downstream tasks with healthcare images using the fine-tuned AI model.

2. The computer-implemented method of claim 1, wherein performing the downstream tasks further comprises performing cancer cell detection from healthcare images to assist a decision-making process of a healthcare provider.

3. The computer-implemented method of claim 2, wherein performing the downstream tasks further comprises updating a medical diagnosis for a patient based on the results of the cancer cell detection.

4. The computer-implemented method of claim 1, wherein computing the activation statistics further comprises conducting a forward pass through the AI model with frozen parameters.

5. The computer-implemented method of claim 1, wherein adjusting the weights of the layers further comprises optimizing model parameters by minimizing an average of layer-wise losses.

6. The computer-implemented method of claim 5, wherein optimizing the model parameters further comprises computing the layer-wise loss as the distance between source data statistics and target data statistics for each batch from the target data.

7. The computer-implemented method of claim 1, wherein fine-tuning the AI model further comprises minimizing an alignment loss that is weighted by respective gradient to parameter norm ratio and a layer-wise loss for the layers of the AI model.

8. A system for adapting artificial intelligence (AI) models to different domains, comprising:
a memory device;
one or more processor devices operatively coupled with the memory device to:
compute activation statistics for an AI model from collected domain data;
adjust weights of layers of the AI model based on the activation statistics by employing metrics of training gradients;
fine-tune the AI model by focusing adaptation intensity to layers with attention mechanism by using a ratio of gradient norm over parameter norm to obtain a fine-tuned AI model; and
perform downstream tasks with healthcare images using the fine-tuned AI model.

9. The system of claim 8, wherein to perform the downstream tasks further comprises to perform cancer cell detection from healthcare images to assist a decision-making process of a healthcare provider.

10. The system of claim 9, wherein to perform the downstream tasks further comprises to update a medical diagnosis for a patient based on the results of the cancer cell detection.

11. The system of claim 8, wherein to compute the activation statistics further comprises to conduct a forward pass through the AI model with frozen parameters.

12. The system of claim 8, wherein to adjusting the weights of the layers further comprises to optimize model parameters by minimizing an average of layer-wise losses.

13. The system of claim 12, wherein to optimize the model parameters further comprises to compute the layer-wise loss as the distance between source data statistics and target data statistics for each batch from the target data.

14. The computer-implemented method of claim 1, wherein to fine-tune the AI model further comprises to minimize an alignment loss that is weighted by respective gradient to parameter norm ratio and a layer-wise loss for the layers of the AI model.

15. A non-transitory computer program product comprising a computer-readable storage medium including program code for adapting artificial intelligence (AI) models to different domains, wherein the program code when executed on a computer causes the computer to:
compute activation statistics for an AI model from collected domain data;
adjust weights of layers of the AI model based on the activation statistics by employing metrics of training gradients;
fine-tune the AI model by focusing adaptation intensity to layers with attention mechanism by using a ratio of gradient norm over parameter norm to obtain a fine-tuned AI model; and
perform downstream tasks with healthcare images using the fine-tuned AI model.

16. The non-transitory computer program product of claim 15, wherein to perform the downstream tasks further comprises to perform cancer cell detection from healthcare images to assist a decision-making process of a healthcare provider.

17. The non-transitory computer program product of claim 15, wherein to compute the activation statistics further comprises to conduct a forward pass through the AI model with frozen parameters.

18. The non-transitory computer program product of claim 15, wherein to adjusting the weights of the layers further comprises to optimize model parameters by minimizing an average of layer-wise losses.

19. The non-transitory computer program product of claim 18, wherein to optimize the model parameters further comprises to compute the layer-wise loss as the distance between source data statistics and target data statistics for each batch from the target data.

20. The non-transitory computer program product of claim 15, wherein to fine-tune the AI model further comprises to minimize an alignment loss that is weighted by respective gradient to parameter norm ratio and a layer-wise loss for the layers of the AI model.
